(19) United States
(12) Patent Application Publication
(10) Pub. No.: US 2013/0338562 A1
(43) Pub. Date: Dec. 19, 2013

(54) NEEDLELESS DIALYSIS GRAFT

(71) Applicant: Jody Ramond Williams, Raytown, MO (US)

(72) Inventor: Jody Ramond Williams, Raytown, MO (US)

(21) Appl. No.: 13/517,595

(22) Filed: Jun. 14, 2012

(51) Int. Cl. A61M 1/14

(52) U.S. Cl. 604/6.16

(57) ABSTRACT

A needleless dialysis graft device eliminates the need to use needles during the dialysis process. The device of the present invention can be implanted into a dialysis patient, allowing the patient to be treated instantly, effortlessly and without pain. The device includes a tubing, placed under the skin that attaches on one side to an artery and on the other side to a vein. An access opening can be fluidly connected to each side of the device and may penetrate to the outside of the skin. The snap caps can be removed from both openings the compression lines are attached, then the locking mechanism is then twisted open to begin blood flow. Once dialysis is completed, the locking cap can be twisted shut, the compression lines removed, and the snap caps replaced on the access openings, saving the time waiting for clotting as is needed with conventional methods.

### BACKGROUND OF THE INVENTION

The present invention relates to dialysis accessories and, more particularly, to a needleless dialysis graft that solves the need to use painful needles during dialysis.

In hemodialysis, the patient's blood is pumped through the blood compartment of a dialyzer, exposing it to a partially permeable membrane. The dialyzer is composed of thousands of tiny synthetic hollow fibers. The fiber wall acts as the semipermeable membrane. Blood flows through the fibers, dialysis solution flows around the outside of the fibers, and water and wastes move between these two solutions. The cleansed blood is then returned via the circuit back to the body.

Typically, large and very painful needles are needle to gain arterial and venous access to perform dialysis. Patients have to wait before dialysis for someone to gain this access and then wait after dialysis for clotting. The result is a painful and time-consuming process for a patient.

In other dialysis methods, dialysis can be performed through other parts of the body by the use of catheters. However, this method can result in infections if the catheters and access points are not kept appropriately clean and sanitary.

As can be seen, there is a need for an improved process for gaining arterial and venous access for repeated dialysis treatments for a patient.

### SUMMARY OF THE INVENTION

In one aspect of the present invention, a needleless dialysis graft comprises a device body configured as a tubular member adapted to connected at a first end to a patient's vein and at a second end to a patient's artery; a first port and a second port fluidly connected with the device body; and a first cap and a second cap removably disposed over the first port and the second port.

In another aspect of the present invention, a needleless dialysis graft comprises a device body configured as a tubular member adapted to connected at a first end to a patient's vein and at a second end to a patient's artery; a first port and a second port fluidly connected with the device body; a first cap and a second cap removably disposed over the first port and the second port; a lip disposed in each of the first port and the second port; and one or more wings on a top outer surface of the first and second caps, wherein the first and second caps are disposed over the first and second ports by a twist lock mechanism.

In a further aspect of the present invention, a method for performing dialysis on a patient comprises implanting a needleless dialysis graft in the patient, the needleless dialysis graft having a device body configured as a tubular member, a first port and a second port fluidly connected with the device body, and a first cap and a second cap removably disposed over the first port and the second port; connecting a first end of the device body to a patient's vein and a second end of the device body to a patient's artery; removing the first and second caps from the first and second ports; and connecting a dialysis machine to the first and second ports.

These and other features, aspects and advantages of the present invention will become better understood with reference to the following drawings, description and claims.

### BRIEF DESCRIPTION OF THE DRAWINGS

FIG. 1 is a perspective view of an arm having a needleless dialysis graft inserted therein, according to an exemplary embodiment of the present invention;

FIG. 2 is a side perspective view of the arm of FIG. 1 with the needleless dialysis graft inserted therein;

FIG. 3 is a cross sectional view taken along line 3-3 of FIG. 1;

FIG. 4 is a perspective view of a needleless dialysis graft device according to an exemplary embodiment of the present invention;

FIG. 5 is a side view of the needleless dialysis graft device of FIG. 4;

FIG. 6 is an exploded perspective view of the needleless dialysis graft device of FIG. 4;

FIG. 7 is a top view of the needleless dialysis graft device of FIG. 4; and

FIG. 8 is a perspective detailed view illustrating a locking mechanism of the dialysis graft device of FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a needleless dialysis graft device that eliminates the need to use needles during the dialysis process. The device of the present invention can be implanted into a dialysis patient, allowing the patient to be treated instantly, effortlessly and without pain. The device of the present invention includes a tubing, placed under the skin that attaches on one side to an artery and on the other side to a vein. An access opening can be fluidly connected to each side of the device and may penetrate to the outside of the skin. A locking cap can be removed from both openings until access is needed during dialysis. Once dialysis is completed, the locking cap can be replaced over the openings, saving the time waiting for clotting as is needed with conventional methods.

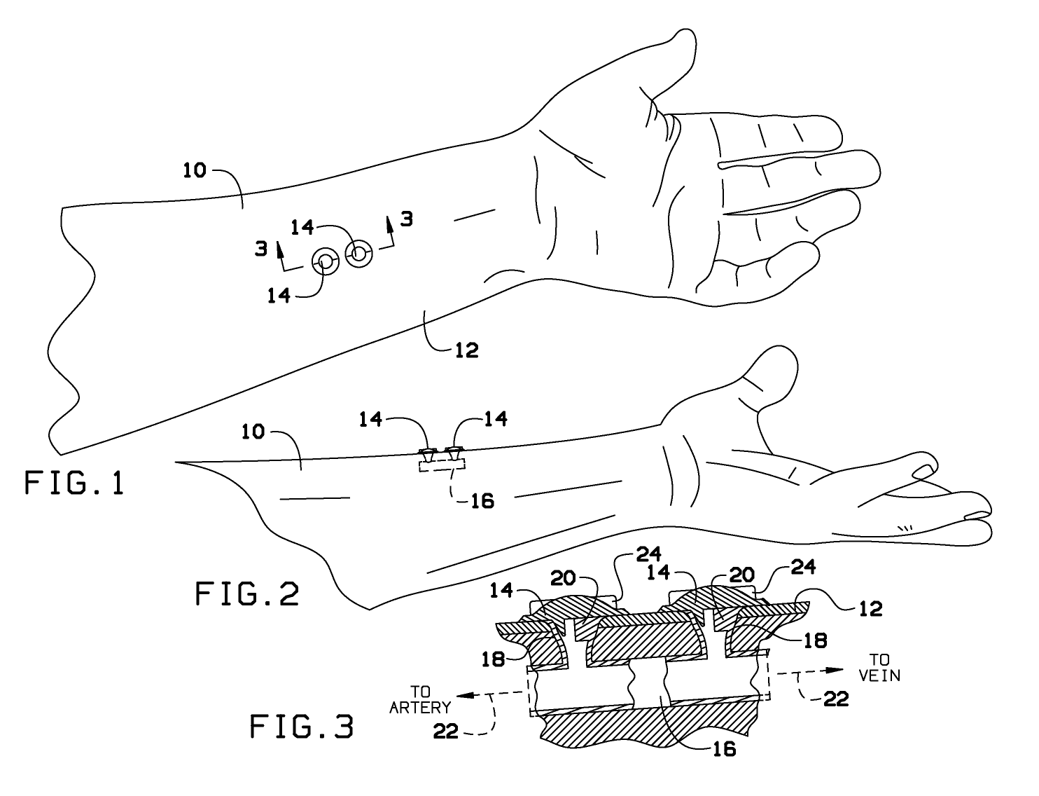

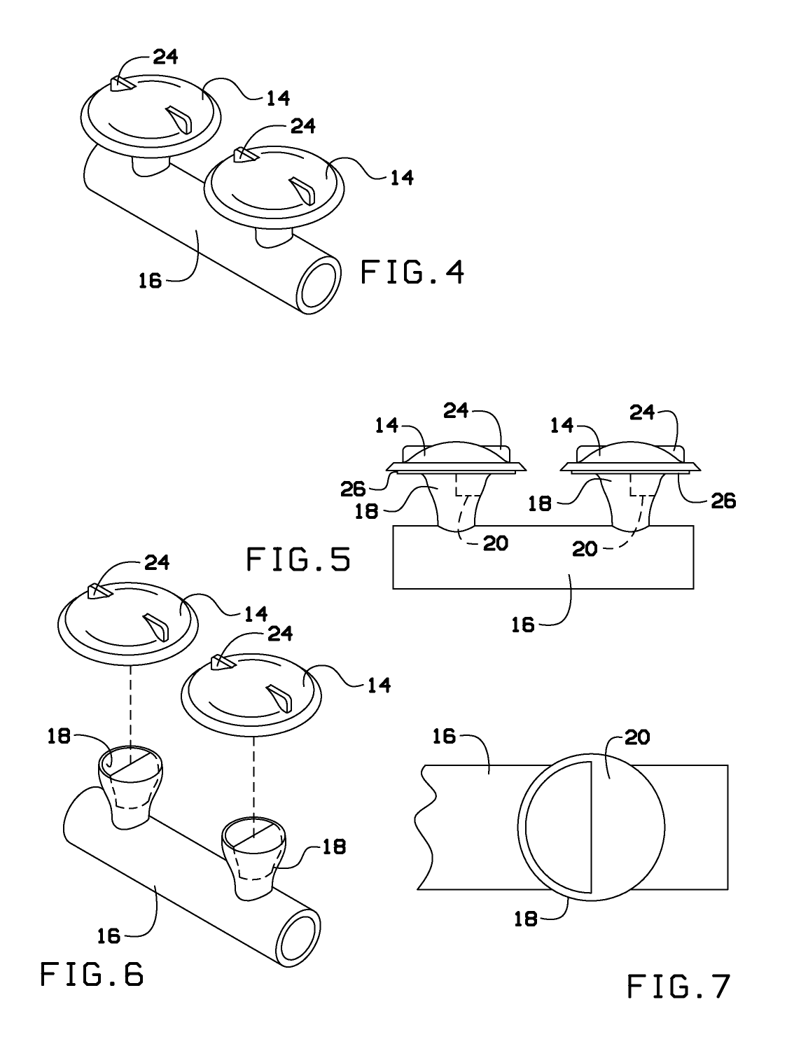

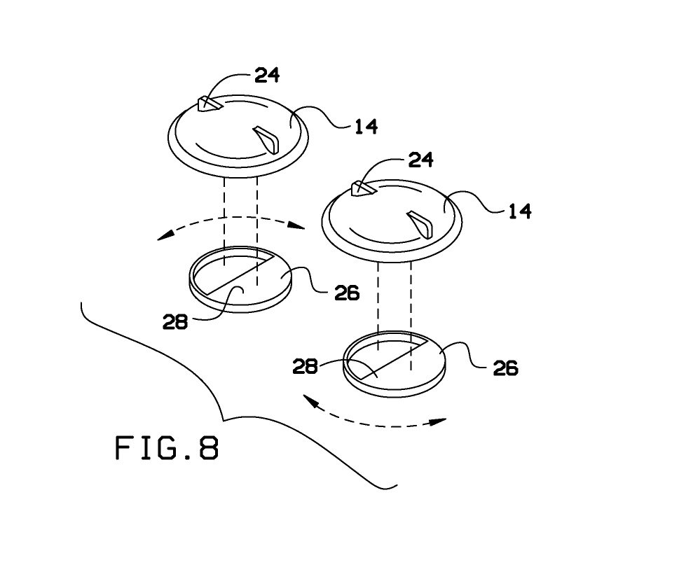

Referring now to FIGS. 1 through 7, a needleless dialysis graft can be disposed such that a device body 16 is disposed under the skin 12 of a patient 10 with two ports 18 extending from the device body 16 to extend outside of the patient's skin 12. The device body 16 may be made from conventional materials, such as medical grade plastic tubing. The device body 16 may connect to a vein at one end thereof, and to an artery at an opposite end thereof.

The ports 18 may fluidly connect with an inside of the device body 16. The ports may include a lip 20 or other connection components to permit a dialysis machine (not shown) to be connected to the ports 18. A solid side 28 of the lip 20 may face outward. A cap 14 may sealingly engage with the ports 18. The cap 14 may include a locking mechanism 26, such as being a twist-lock cap. One or more wings 24 may be disposed on the top of the caps 14 to help in removal and application of the caps 14 on the ports 18.

The port 18 adjacent to the device body 16 at an artery connection may receive blood from the body and deliver it to the dialysis machine. The port 18 adjacent to the device body 16 at a venous connection may receive treated blood from the dialysis machine and deliver that blood back to the body via the needleless dialysis graft.

To use the device, one simply needs to remove the fitted caps 14 on top of the ports 18, install (by twisting, for example) the compression line from the dialysis machine to the device and attach the next section of line from the dialysis machine to the dialysis machine and begin dialysis. When dialysis is complete, the user can simply remove the lines and cap off the ports 18 until next time.

While the above description discusses the use of medical grade plastic for the device body, other materials may be used, as appropriate, to make the various components of the needleless dialysis graft of the present invention. The size and number of needleless dialysis grafts installed in a patient can vary. For example, smaller devices may be installed in multiple access locations on a patient. While the figures show the device disposed in a patient's arm, the device may be installed in various locations on the patient's body.

It should be understood, of course, that the foregoing relates to exemplary embodiments of the invention and that modifications may be made without departing from the spirit and scope of the invention as set forth in the following claims.

What is claimed is:

1. A needleless dialysis graft comprising:
a device body configured as a tubular member adapted to connected at a first end to a patient's vein and at a second end to a patient's artery;
a first port and a second port fluidly connected with the device body; and
a first cap and a second cap removably disposed over the first port and the second port.

2. The needleless dialysis graft of claim 1, further comprising a lip disposed in each of the first port and the second port.

3. The needleless dialysis graft of claim 1, wherein the first and second caps are disposed over the first and second ports by a twist lock mechanism.

4. The needleless dialysis graft of claim 3, further comprising one or more wings on a top outer surface of the first and second caps.

5. A needleless dialysis graft comprising:
a device body configured as a tubular member adapted to connected at a first end to a patient's vein and at a second end to a patient's artery;
a first port and a second port fluidly connected with the device body;
a first cap and a second cap removably disposed over the first port and the second port;
a lip disposed in each of the first port and the second port; and
one or more wings on a top outer surface of the first and second caps, wherein
the first and second caps are disposed over the first and second ports by a twist lock mechanism.

6. A method for performing dialysis on a patient, the method comprising:
implanting a needleless dialysis graft in the patient, the needleless dialysis graft having a device body configured as a tubular member, a first port and a second port fluidly connected with the device body, and a first cap and a second cap removably disposed over the first port and the second port;
connecting a first end of the device body to a patient's vein and a second end of the device body to a patient's artery;
removing the first and second caps from the first and second ports; and
connecting a dialysis machine to the first and second ports.

7. The method of claim 6, further comprising:
removing the dialysis machine from the first and second ports when dialysis is complete; and
re-attaching the first and second caps to the first and second ports.

8. The method of claim 6, further comprising twisting the first and second caps to remove and attach them from the first and second ports.
